(19) United States
(12) Patent Application Publication
(10) Pub. No.: US 2010/0047855 A1
(43) Pub. Date: Feb. 25, 2010

(54) METHOD FOR DETERMINING THE QUANTITY OF MICROBIOLOGICAL OBJECTS DURING CULTIVATION THEREOF

(76) Inventors: Ihor Mikhajlovich Kushnir, Lvov (UA); Ihor Yaroslsvovich Kotsyumbas, Lvov (UA); Alekaander Ivanovich Bilyy, Lvov (UA); Vasilly Bogdanovich Getman, Lvov (UA); Rostislav Alekasndrovich Bilyy, Lvov (UA)

Correspondence Address: Aleksandr Smushkovich, Pob 140505, Brooklyn, NY, 11214, US

(21) Appl. No.: 12/309,433

(22) PCT Filed: Jul. 23, 2007
(86) PCT No.: PCT/UA2007/000044

§ 371 Date: Jun. 29, 2009

(30) Foreign Application Priority Data
Jul. 25, 2006 (UA) A 2006 08347

(51) Int. Cl. C12Q 1/06

(52) U.S. Cl. 435/39

(57) ABSTRACT

A method for determining the quantity of microbiological objects during the cultivation thereof comprises determining a modification of studied microorganism cells' population, measuring the morphological composition thereof by determining the cells' size distribution in a liquid medium according to intensity changes of the light dispersed thereby. The method comprises probing a liquid flow by monochromatic coherent light, recording signals relating to the interaction between the probing radiation and the cells by measuring amplitudes and durations of impulses of the light dispersed by medium particles, plotting functions, according to measurement results, as two-dimensional distributions of normalized values of the amplitudes and durations in the form of statistic parameters of the dispersed light intensity. According to the functions, the size distribution of the cells and decomposition products of the liquid nutrient medium during the cultivation thereof are obtained. The invention can be applied for medical diagnostics and for control of biotechnological processes.

### CROSS-REFERENCE TO RELATED APPLICATIONS

This application is a U.S. national phase application of a PCT application PCT/UA2007/000044 filed on 23 Jul. 2007, published as WO2008/013512, whose disclosure is incorporated herein in its entirety by reference, which PCT application claims priority of a Ukrainian patent application UA2006/08347 filed on 25 Jul. 2006.

### FIELD OF THE INVENTION

This invention relates to the optical methods of determining the amount of changes of microbiological objects such as cells and cell lines, bacteria, yeasts, fingi in the process of their cultivation and can be applied for diagnostic aims in medicine and for control of biotechnological processes.

### BACKGROUND OF THE INVENTION

It is known that determination of the amount of microbiological objects in the process of their cultivation is based on the so-called growth curves, functional time dependence of cell quantities in the process of their growth. For bacterial cells four areas can be distinguished on the growth curve, particularly lag-phase, exponential phase (or log phase), stationary phase, and death phase [Albert G. Moat, John W. Foster, Michael P. Spector. Microbial Physiology. 4th ed.-New York.-Copyright by Wiley-liss, Inc.-2002-714 pp]. Quantitative estimation of bacteria cell development in the process of their cultivation is usually carried out in an exponential phase (Baranyi J., Pin C. Estimating bacterial growth parameters by means of detection times./Apl. Environmen. Microbiol.-1999.-Vol. 65. 732-736). The method of determining the amount of microbiological objects, based on a turbidity change of liquid culture medium in the process of their cultivation is known (Microbiology Reader Bioscreen C and Software Research Express-Microbiological Growth Curves-http://www.bionewsonline.com/b/5/growth_curves.htm).

The turbidity change of culture medium is measured by an optical spectrophotometer. The turbidity curve represents a graphical dependence of optical density of a solution upon time. In this case, the optical density consists of two components: a turbidity of solution determined by any change in the amount of microbiological cells in the process of their cultivation, and a turbidity of solution, determined by any change of physical and chemical parameters of the culture medium. By mathematical extrapolation of the turbidity curve, it is possible to determine a general concentration of cells, a cell doubling time in the process of their proliferation, and also a change of these parameters of microbiological cells in the process of their cultivation.

Limitations of the aforementioned method for determining the amount of microbiological objects includes impossibility to determine the optical density of solution, conditioned solely by changes of the amount of microbiological cells in the process of their cultivation. Accuracy of this method is determined by the necessity of reliable registration of the optical density of solution in the interval of 0.3-1.0, which corresponds to the concentration of bacterial cells of 10¹²-10¹⁴ per m³ in the analyzed solution. This results in considerable measuring errors for solutions with the concentration of cells less than 10¹² per m³.

Another method of determining the amount of microbiological objects is known, which is based on calculations of dielectric permeability by measuring impedance spectrums of a liquid culture medium in the process of their cultivation [US Patent US20040009572 from Jan. 15, 2004, IPC C12M1/00. Apparatus for the analysis of microorganisms growth and procedure for the quantification of microorganisms concentration; K. G. Ong, J. Wang, R. S. Singh, L. G. Bachas, C. A. Grimes/Monitoring of bacteria growth using and wireless, remote query resonant-circuit sensor: application to environmental sensing//Biosensors & Bioelectronics 2001-Vol. 16-P. 305-312]. The dielectric permeability of the environment is a complex parameter, consisting of real and imaginary parts, calculated based on frequency dependences of active and reactive resistances in the impedance spectrums. The real part is calculated on the basis of the values of resonance frequency, corresponding to the maximum of frequency dependence of resistance in the impedance spectrums; and the imaginary part is calculated using the values of frequency whereat the reactance in the impedance spectrums equals to zero.

The process of measuring impedance spectrums comprises the following steps. By immersing two or three electrodes in a liquid culture medium with cultivated microbiological cells, a direct current voltage developed therein is measured. With the increase of cells concentration, the complex dielectric permeability of medium also increases, causing a change in the capacitance of a condenser placed between the submerged electrodes, and is accompanied by displacements of the resonance frequency and of the zero reactance value frequency.

By measuring the new values of the indicated frequencies, it is possible to evaluate the change in the cell culture concentration during their cultivation. (US Patent Application Publication US20040009572 Apparatus for the analysis of microorganisms growth and procedure for the quantification of microorganisms concentration; K. G. Ong, J. Wang, R. S. Singh, L. G. Bachas, C. A. Grimes/Monitoring of bacteria growth using and wireless, remote query resonant-circuit sensor: application to environmental sensing//Biosensors & Bioelectronics 2001-Vol. 16-P. 305-312).

A disadvantage of the aforementioned method for determination of the amount of microbiological objects is impossibility to register cells in solutions with a concentration less than 10¹³ cells/m³. Besides, external factors greatly influence the accuracy of the measurements. Such factors are: polarization of the electrodes in the process of measurement, formation of gas bubbles on the surface of electrode that causes changes of the electrode potential and of the capacitance of the condenser situated between the electrodes.

There is known a method of determining the amount of microbiological objects, which is based on the analysis of intenseness of changes in the lighting of fluorescent marker molecules, which marker molecules are bound with fragments of the molecular structure of the cell.

The external membrane of the cell accommodates approximately 10⁵ biological receptors; the fluorescent marker molecules interact with the receptors that allows registering the one to register microbiological cells. Examples of fluorescent marker molecules are: fluocerine diacetate, Alexa Fluor and Fluorescein of Oregon Green™ (PCT Application Publication WO02/057482 as of Jan. 17, 2002 Method for differential analysis of bacteria in sample); methylumbelliferone (US Patent Application Publication US2003/0138906 filed Jul. 24, 2003); Fluorescence test for measuring heterotrophic bacteria in water, and lots of other compounds (U.S. Pat. No. 6,632,632 issued Oct. 14, 2003; Rapid method of detection and enumeration of sulfide-producing bacteria in food products. PCT Application Publication WO99/10533 filed on Apr. 17, 1999; Rapid detection and identification of microorganisms).

Estimation of the quantity of microorganisms cells in the process of their cultivation is possible by measuring the lighting intensity of marker molecules and is provided as follows below. (Brian J. Mailloux, Mark E. Fuller. Determination of in situ bacterial growth rates in aquifers and aquifer sediments.//Applied and Environmental Microbiology.-2003.-Vol. 69, No. 7.-P. 3798-3808).

In the process of cells cultivation with an increase of their concentration, the average lighting intensity of marker molecules declines. Every time when the cell concentration is doubled, the lighting intensity of marker molecules decreases twice, taking into account the background lighting.

Thus, in the process of fluorescence analysis, it is assumed that if the lighting intensity of marker molecules decreases by a half, then the number of cells has doubled. At the beginning of measurement, the average lighting intensity of the initial cells is Iₚ, and the average background lighting intensity of the cells, not containing fluorescent marker molecules is I_{b}, taking into account that at a moment of time t an average lighting intensity of marker cells is Iₜ where the initial cell concentration is C₀ and their concentration at any moment of time t is equal to Cₜ, then the average lighting intensity of marker molecules Iₜ is determined by the formula:

I^{t}=(Iₚ−I_{b})Cₜ/C₀+I_{b} (1)

By measuring the lighting intensity changes one can calculate concentrations of microorganism cells in the process of their cultivation, and the time needed for doubling the cell population.

Shortcomings of the mentioned method are: complexity and low cost efficiency caused by the use of expensive reagents and high-tech measurement equipment. Besides, the list of available fluorescent marker molecules is limited, their cost is high, and in most cases they are harmful for the human organism.

The closest to the proposed invention is a method for determination of size distribution of the suspended particles in the stream of liquid, described in Patent of Ukraine UA42971C issued Dec. 15, 2003. Method and device for determination of size distribution of suspended particles in the stream of liquid.

The method is based on registration of fluctuations of the intensity of light dispersed by particles, wherein the registration is provided by means of statistical collection of changes of impulses' amplitude and duration for the particles of given size, by building of a correlation function F(U,t) on the basis of these measurements, which expresses statistical descriptions of light intensity scattered by the particles, and by obtaining the size distribution of particles by solving Fredholm's first-type integral equation:

$$F ( U , t ) = {\int }_{{r}_{max}}^{{r}_{min}} K ( U , t , r ) n ( r ) d r , \qquad ( 2 )$$

where rₘᵢₙ and rₘₐₓ is a lowest and highest limits of the range of particles sizes to be registered;
n(r)dr—is a function of particles distribution depending on size;
K(U,t,r)—is a distribution function of values of amplitudes and durations of the registered impulses of light dispersed by calibration (reference) particles, which is the result of any previous probing by monochromatic coherent light of a liquid stream containing polymer latex microparticles of a predetermined size and a known refraction index.

A disadvantage of this method is considerable errors during determination of quantities of microbiological objects caused by the appearance of foreign particles—products of disintegration of the culture medium in the process of cultivation of the microbiological objects being studied.

The current invention is aimed to improve methods of determination of size distribution of suspended particles in the stream of liquid by independent registration of particles in the process of cultivation of microbiological objects in the liquid medium containing studied cells, and in the liquid medium without studied cells. This approach allows providing the quantitative determination of changes of microbiological objects in the determined size interval during the cultivation process. This also allows eliminating errors, caused by the appearance of foreign particles, namely products of disintegration of the liquid medium in the process of cultivation of studied microbiological objects.

### SUMMARY OF THE INVENTION

The above mentioned problem is solved by the inventive method for determining the quantity of microbiological objects during their cultivation comprising the following steps: supplying a substantially constant speed stream of liquid containing the microbiological objects; probing the stream by means of monochromatic coherent light radiation; calibrating by means of plotting a two-dimensional distribution of registered impulses in a predetermined volume of spherical particles with a known index of refraction and having a radius r as a function of K(U,t,r) depending on values of normalized amplitudes U and a duration t of dispersed radiation impulses; probing the stream of liquid with the studied microbiological objects by the same monochromatic coherent light in the predetermined calibrated volume; registering light impulses dispersed by the probed particles at a predetermined angle in an interval from 1 to 360 degrees; determination of a function n(r) of size distribution of the studied particles based on the registration of probed particles impulses; sorting the registered impulses for plotting a two-dimensional distribution of their amplitudes and duration in sub-ranges with limits chosen for calibration of the probed particles; normalization of numeral values of the amplitudes and durations of impulses in each of the sub-ranges; based on the normalized values plotting a function F(U,t), which determines a size distribution of the studied objects in a size range from rₘᵢₙ to rₘₐₓ as:

$$F ( U , t ) = {\int }_{{r}_{max}}^{{r}_{min}} K ( U , t , r ) n ( r ) d r , \qquad ( 3 )$$

according to the invention, cultivating selected for research microbiological objects in a liquid nutrient medium, determination of time-dependent changes of the quantity of microbiological objects and background particles in the medium, for which purpose diluting the liquid medium with studied microbiological objects and diluting the liquid medium without them in equal proportions in a physiological solution of sodium chloride, 5% glucose solution or de-ionized water; separate determination of a general size distribution of microbiological objects and background particles in a high-pure liquid, which contains microbiological objects, and a general size distribution of background particles in the high-pure liquid wherein the microbiological objects are absent; determination of a quantity of microbiological objects in a predetermined size range by means of calculation of the quantity of particles in the medium with cells and without them; determination of a relative content of microbiological objects in the predetermined size range by computing a ratio of a share of microbiological objects in the predetermined size range to the total quantity of microbiological objects, plotting time relations of changing of quantity of microbiological objects and their relative contents in the predetermined size range during the cultivation process.

It is known from relevant literature that the quantitative changes of microbiological objects in the process of their cultivation are most active in the exponential growth phase, wherein all cells are divided constantly by the binary fission, and cell proliferation takes place according to a geometrical progression.

During this growth phase, the speed of increase of cell number is permanent, and is determined by the time of doubling cell population (i.e. appearance of a new cell generation). Depending on the cell type, the time of appearance of a new cell generation is in the range from 12 minutes to 24 hours or even more. The frequency of appearance of a new cell generation depends on many factors: concentrations of nutrients, conditions of incubation such as temperature, pH, rate of mixing. Reliability of registration of microorganisms by an instrumental device depends on a lower threshold of cell concentration in the solution, which can be detected by the device.

For a method based on changes in turbidity of a liquid medium, the lower registration threshold is 10¹² cells/m³.

A method, based on the calculations of dielectric conductivity related to impedance spectrums of the liquid nutrient medium has the lower registration threshold of 10¹³ cells/m³.

Exemplarily, time of appearance of a new cell generation is equal to 24 minutes, and an initial concentration of cells in the solution is 10⁸ cells/m³, the reliable registration of this type of cells by the indicated methods is possible at least 5.5 hours after the beginning of their growth.

Currently, most sensitive are fluorescent methods of analysis are based on the analysis of fluorescence intensity change resulting from interactions of marker molecules with microorganisms.

It is known that for the detection of fluorescence, the optimum number of molecules-markers per cell is from 1000 to 100000. Therefore the fluorescent methods of analysis under a certain selection of molecules-markers allow registering 10⁷ cells per m³.

A disadvantage of all the mentioned methods is the possibility of registering just the full quantity of microorganisms dividing them based on morphological features.

The authors of the present invention propose to measure the morphological content of cells by determination of a size distribution of microorganisms cells in a liquid nutrient medium based on changes of light dispersed by the cells for detection of quantity changes of microorganisms during their cultivation.

According to Patent of Ukraine UA 42971C as of 15 Dec. 2003 ‘Method and device for determination of microparticles' size distribution in a liquid flow’, in a method of determination of sizes distribution of suspended particles in a liquid stream, the particles size is estimated based on the intensity of light dispersed by the particles, by measuring two parameters: an amplitude and a duration of pulses dispersed by the particles, further statistical accumulation of the indicated parameters, and finding the size distribution of particles based on data of a statistical set by solving Fredholm's first order integral equation.

The process of cell cultivation in the liquid medium is accompanied with changes in size distribution of particles resulting from both the appearance of a new cells generation in the solution during the reproduction process, and from products of destruction of the liquid medium.

For distinguishing microorganisms from the total quantity of particles, the authors propose a calculation of the size distribution separately for background particles, and for background particles plus microorganisms cells in a liquid nutrient medium by measuring two parameters: an amplitude and a duration of light impulses dispersed by the particles. In this case, the liquid mediums with and without microorganisms are cultivated simultaneously and identically. From statistical measurings' of data of the amplitude and duration of impulses, by solving two Fredholm's first type integral equations, one can calculate the actual amount of microorganisms cells of a certain morphological content, and determine their relative content to the total amount of cells, and also obtain time-dependent data for these parameters.

### BRIEF DESCRIPTION OF THE DRAWINGS

FIG. 1 illustrates a graphic of time dependence of particles of E. coli bacterial cells' concentration and of products of disintegration of liquid medium No. 1 (meat infusion broth) (curve (*)) in 0.9% sodium chloride solution, according to an embodiment of the present invention. The upper (□) and lower (∘) curves determine the limits of a confidence interval.

FIG. 2 illustrates a graphic of time dependence of particles of E. coli bacterial cells' concentration and of products of disintegration of liquid medium No. 3 (enriched medium for detection of bacteria Enterobacteriaceae) (curve (*)) in 0.9% sodium chloride solution are presented. Upper (□) and lower (∘) curves determine the limits of a confidence interval.

FIG. 3 illustrates a graphic of size distribution of particles of E. coli bacterial cells' and products of disintegration of liquid medium No. 3 (enriched medium for detection of bacteria Enterobacteriaceae) in the 0.9% sodium chloride solution according to an embodiment of the present invention, size range is 0.2-2.0 μm. A curve (∘) represents a suspension of particles, containing the bacterial E. coli cells from a non sterile vessel; a curve (+) represent a suspension of particles, containing the bacterial E. coli cells from a sterilized vessel.

FIG. 4 illustrates a graphic of size distribution of particles of E. coli bacterial cells' and products of disintegration of liquid medium No. 3 (enriched medium for detection of bacteria Enterobacteriaceae) in 0.9% sodium chloride solution according to an embodiment of the present invention, size range is 0.2-2.0 μm. A curve (∘ drawn with small circles) represents a suspension of particles, containing the bacterial E. coli cells from a non sterilized vessel; a curve (□ drawn with small squares) represent a suspension of particles, containing the bacterial E. coli cells from a non sterilized vessel after 24 hours of incubation.

FIG. 5 illustrates a graphic of size distribution of particles of E. coli bacterial cells' and products of disintegration of liquid medium No. 3 (enriched medium for detection of bacteria Enterobacteriaceae) in 0.9% sodium chloride solution according to an embodiment of the present invention, size range is 0.2-2.0 μm. A curve (*) represents a suspension of particles, containing the bacterial E. coli cells from a sterilized vessel; a curve (+) represent a suspension of particles, containing the bacterial E. coli cells from a sterilized vessel after 24 hours of incubation.

FIG. 6 illustrates a graphic of the relative contents of particles—products of disintegration of liquid medium No 3 (enriched medium for detection of bacteria Enterobacteriaceae) in 0.9% sodium chloride solution according to an embodiment of the present invention, size range is 0.2-2.0 μm. Curve 1 represents suspension of particles at the beginning of cultivation process, curve 2 represents suspension of particles after 4 hours of incubation, curve 3 represents suspension of particles after 5 hours of incubation, curve 4 represents suspension of particles after 6 hours of incubation.

FIG. 7 illustrates a graphic of the relative contents of particles—bacterial E. coli cells and products of disintegration of liquid medium No 3 (enriched medium for detection of bacteria Enterobacteriaceae) in 0.9% sodium chloride solution according to an embodiment of the present invention, size range is 0.2-2.0 μm. Curve 1 represents suspension of particles at the beginning of cultivation process, curve 2 represents suspension of particles after 4 hours of incubation, curve 3 represents suspension of particles after 5 hours of incubation, curve 4 represents suspension of particles after 6 hours of incubation.

FIG. 8 illustrates a graphic of normalized distribution of relative changes of particles—bacterial E. coli cells in relation to data at the beginning of cultivation process in a liquid medium No 3 (enriched medium for detection of bacteria Enterobacteriaceae) according to an embodiment of the present invention, size range is 0.2-2.0 μm. Curve 1 represents normalized distribution of bacterial E. coli cells at the beginning of cultivation process; curve 2 represents normalized distribution of bacterial E. coli cells after 4 hour of incubation in relation to normalized initial distribution of these cells at the beginning of cultivation process; curve 3 represents normalized distribution of bacterial E. coli cells after 5 hour of incubation in relation to normalized initial distribution of these cells at the beginning of cultivation process, curve 4 represents normalized distribution of bacterial E. coli cells after 6 hour of incubation in relation to normalized initial distribution of these cells at the beginning of cultivation process.

FIG. 9 illustrates a graphic of relative content of the Mucor racemosus mold particles and products of disintegration of liquid Saburo medium in 5% glucose solution according to an embodiment of the present invention after 2 days of cultivation, size range is 0.2-2.0 μm.

### DESCRIPTION OF PREFERRED EMBODIMENTS OF THE INVENTION

The method of determination of quantities of microbiological objects in the process of their cultivation comprises: supplying studied microbiological objects in a stream of liquid with a constant speed; passing a laser beam through the stream of liquid with studied microorganism cells so that a cross-section of the beam in the place of its intersection with the cells is greater the cross-section of studied cells, and also that only one cell at a certain moment of time can intersect the laser beam; and further determination of the distribution of particles' sizes in the process of their cultivation.

A procedure of calibration of a device for registration of particles is preliminary done for this purpose. It comprises a plotting of a function that determines a two-dimensional distribution of registered impulses in a predetermined volume of spherical particles with known refraction index. For this reason, all particles in the stream of liquid intersect a probing laser beam during a predetermined period of time, the particles disperse the beam radiation, whose light impulses are registered with regard to their amplitude and duration.

The registered impulses are sorted based on their amplitudes and durations, for this reason the range of impulses' amplitudes is divided into ‘m’ sub-ranges, wherein the lowest boundary of the amplitude range is limited by the noise voltage level of the photo-electric registration system, and the upper boundary of the amplitude range is limited by a saturation voltage of the input amplifier.

The range of durations is divided into ‘n’ sub-ranges and is limit by its highest and lowest values, corresponding to the time needed for a cell to pass the maximal and minimal cross-sections of the laser beam.

The values of amplitudes and durations of impulses in the indicated sub-ranges are normalized in accordance with a certain algorithm. Characteristics of the two-dimensional distribution of sizes of particles are plotted for each of the mentioned sub-ranges. The procedure of determination of the cells sizes distribution in the liquid flow comprises: probing the stream of liquid with the studied objects by the same monochromatic coherent light and in the same volume, which were used for the calibration; based on the measurement results, plotting a function for determination of statistical properties of the light dispersed by the cells; mathematical decomposition of this function into components with respect to the amplitudes and durations of the registered impulses for each of the sub-ranges, based on the data obtained during the calibration procedure.

For exclusion of a dependence of the obtained results upon the time of measuring, a normalization operation is conducted. In case when the measuring procedure includes only one operation of measuring, the normalization operation consists of division of the numeral value in each sub-range by the sum of the numeral values in all the sub-ranges.

In case when the measuring procedure includes at least two operations of measurement: e.g. the basic measurement and the verifying measurement, the normalization operation consists of division of the numeral values in each sub-range for the basic and verifying measurement operations by the time of each measurement; further subtraction in every sub-range of the numeral values, obtained for the verifying operation of measurement, from the numeral values of sizes, obtained for the basic operation of measurement; division of the thus obtained values in each sub-range by the sum of numeral values of amplitudes and durations of the registered impulses, obtained during the calibration procedure; arranging a matrix of type A(m,n), having an element a_{if}(m,n,) equal to the normalized values of amplitude and duration of the registered impulses of dispersed light for a particular spherical particle of a predetermined size and index of refraction; based on the normalized data of numeral values of amplitudes and durations of the registered impulses obtained during the measurement procedure, arranging a matrix of type B(m,n,), having an element bᵢⱼ(m,n) equal to the normalized values of amplitude and duration of the registered impulses. Determination of the particles' sizes distribution in the flow of liquid is done by solving the equation:

|B(m,n,)−Σφ_{I}A(m,n,)|=0 (4)

where
i—is a number of the spherical particles of a predetermined size and index of refraction chosen for calibration,
j—is a sequence number of a matrix element and j≦i,
φᵢ—is a constant value, in the range of from 0 to 1.

Determination of the quantity of microbiological objects in the process of their cultivation is achieved by successive measurements of time dependences of the light-dispersing parameters of the liquid medium, in which medium the studied microorganism cells are cultivated, with the cell objects, and without them.

An increase of accuracy of the measurements is achieved by multiple dilutions in identical proportions of the liquid medium with cells and without them in one of the following high-pure liquids: 0.9% sodium chloride solution, 5% glucose solution, or de-ionized water.

The measurement procedure comprises the following steps: sequential determination of a general size distribution of particles in one of the above enumerated high-pure liquid containing the liquid medium with cell objects and without them. The quantity of microbiological objects in a predetermined size range is obtained by subtraction of the number of particles in the medium from the number of particles in the medium with cells. Determination of the relative content of microbiological objects in the predetermined size range is carried out by calculation of the portion of microbiological objects in the predetermined size range to the total number of microbiological objects. Time-dependences of changes of the quantity of microbiological objects and their relative content in the predetermined size range during the cultivation process are calculated based on the obtained data.

The results of measurements during the determination of the particles size distribution are of statistical origin, and statistical analysis is performed for estimation of their reliability. For this purpose, a measurement of each sample of the liquid medium is performed at least 5 times with further statistical evaluation of the obtained results by determination of mean values and standard deviations a of the measurement results. During the reliability estimation of the measurement results, they stay in the limits of a confidence interval Δ related to the mean value, which is calculated through the formula:

Δ=t(s,P)σ, (5)

wherein: t—is Student's distribution coefficient for a number of measurements s and a confidence probability P. For further calculations, the value of confidence probability is set at P=0.95, meaning that an interval ū−Δ<u<ū+Δ includes at least 95% of the measurement results.

### EXAMPLES OF INDUSTRIAL APPLICABILITY

Industrial applicability of the invention can be demonstrated with the following examples.

### Example 1

The study of Escherichia coli bacterial cells in a liquid medium No 1 (meat infusion broth) was conducted in accordance with requirements of the State Pharmacopoeia of Ukraine. For this purpose 10.0 g of dry fermentative peptone and 5.0 g of sodium chloride are dissolved under slow heating in a flask with 1 liter of meat water, 1.0 g of glucose is added for setting the pH level to be 7.3±0.2 after sterilization, and is simmered for 1 minute. The medium is filtered through a membrane filter with 0.2 μm pores, then sterilized in a steam sterilizer at 121° C. for 15 minutes. Escherichia coli bacterial cells are put into the medium at an initial concentration of 3300 cells/ml of medium. The so prepared solution of bacterial E. coli cells is bottled into sterile test tubes, 5 cm³ each, the tubes are closed with wadding-gauze corks, incubated in a thermostat at 30° C. Measurements of the dispersed light intensity by particles in the medium are carried out 7 times with an interval of 1 hour. For this reason, a preliminary specimen preparation is performed: a specimen of liquid medium with bacterial cells after a fixed incubation time in a test tube is diluted 1:100 in sterile 0.9% sodium chloride solution in a 200 ml capacity, closed with a rubber cork with an aluminum rim. (0.9% sodium chloride solution is serially produced by the pharmaceutical industry. The prepared specimen of E. coli cells is transferred by a sterile 20 ml syringe into a syringe pump and measurements of intensity change of the light dispersed by particles are performed using a PRM-6 device, described in Patent of Ukraine UA 42971C from Dec. 15, 2003 ‘The method and device for determination of size distribution of suspended particles in the liquid fluid’ hereby entirely incorporated by reference. The PRM-6 device is preliminary calibrated using spherical polysterene latexes with refraction index n=1.56. Measurements of every specimen are performed 5-6 times with further statistical analysis of the measurement results. Between the measurements of every specimen, the hydraulic passway and optical cuvette of the aforesaid device are washed with de-ionized water. For a chosen number of measurements and a confidence probability P=0.95, the Student's coefficient and limits of the confidence interval relative to a mean value are determined using formula (5).

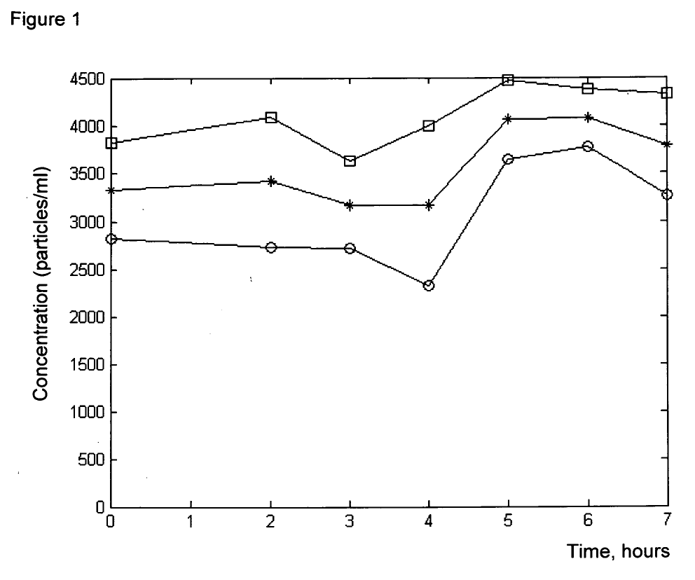

FIG. 1 illustrates a time dependence of particles of E. coli bacterial cells' concentration and of the products of disintegration of the liquid medium No. 1 in the 0.9% sodium chloride solution. The upper (□) and lower (∘) curves determine the limits of confidence interval in relation to a mean value, determined by t-values t(5; 0.95)=2.77 and t(6; 0.95)=2.57. From the presented time dependence of the particles concentration change, one can conclude that within the error limits an increase of the number of particles is not observed during the incubation in the liquid medium.

### Example 2

The study of Escherichia coli bacterial cells in a liquid medium No 3 (enriched medium for detection of bacteria Enterobacteriaceae) was conducted in accordance with requirements of the State Pharmacopoeia of Ukraine. For this purpose 10.0 g of dry fermentative peptone, 7.5 g of Na₂HPO₄ and 2.5 g of KH₂PO₄ are dissolved under slow heating in a flask with 1 liter of meat water, 10.0 g of glucose, 8 ml of 1% phenol red solution and 3 ml of malachite green solution are added for setting the pH level to be 7.3±0.2 after sterilization. The medium is filtered through a membrane filter with 0.2 μm pores, then sterilized in a steam sterilizer at 121° C. for 15 minutes. Escherichia coli bacterial cells are put into the medium at an initial concentration of 3000 cells/ml of the medium. The prepared solution of bacterial E. coli cells is bottled into sterile test tubes, 5 cm³ each, the tubes are closed with wadding-gauze corks, and incubated in a thermostat at 40° C. Measurements of the intensity of light dispersed by particles in the medium are carried out 7 times with an interval of 1 hour.

For this reason the preliminary specimen preparation is performed: a specimen of the liquid medium with bacterial cells after fixed incubation time in a test tube is diluted 1:100 in a sterile 0.9% sodium chloride solution in a 200 ml capacity, closed with a rubber cork with an aluminum rim. (0.9% sodium chloride solution is serially produced by the pharmaceutical industry). The prepared specimen of E. coli cells is transferred by a sterile 20 ml syringe into a syringe pump and measurements of the intensity change of light dispersed by the particles are performed using the PRM-6 device, described in Patent of Ukraine UA 42971C. The PRM-6 device is preliminary calibrated using spherical polysterene latexes with a refraction index n=1.56. Measurements of every specimen are performed 5-6 times with the further statistical analysis of the measurement results. Between the measurements of every specimen the hydraulic passway and optical cuvette of the device are washed with de-ionized water. For a chosen number of measurements s and confidence probability P=0.95, Student's coefficient and the limits of a confidence interval relative to a mean value are determined using formula (5).

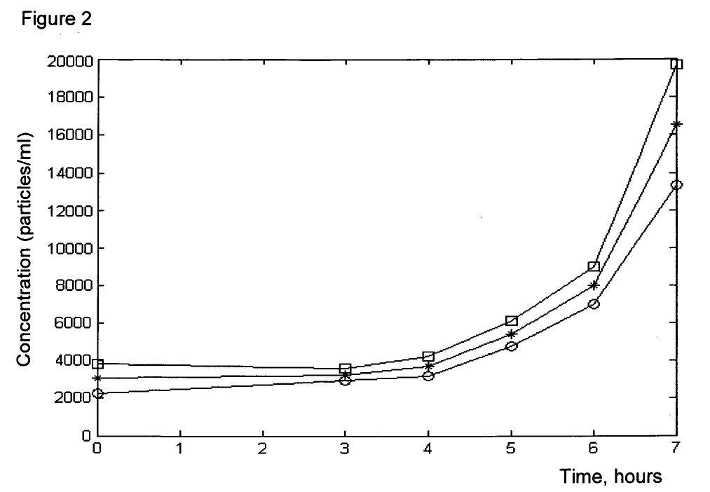

On FIG. 2, a time dependence of particles of E. coli bacterial cells' concentration and a time dependence of products of disintegration of the liquid medium No. 3 (curve (*)) in 0.9% sodium chloride solution are presented. The upper (□) and lower (∘) curves determine the limits of confidence interval in relation to the mean value, determined by t-values (5; 0.95)=2.77 and t(6; 0.95)=2.57. From the presented time dependence of particles concentration change, one can conclude that within the error limits an increase in the number of particles is observed after 5 hours of incubation in the liquid medium.

### Example 3

The study of Escherichia coli bacterial cells in a liquid medium No 3 (enriched medium for detection of bacteria Enterobacteriaceae) was conducted in accordance with requirements of the State Pharmacopoeia of Ukraine. For this purpose 10.0 g of dry fermentative peptone, 7.5 g of Na₂HPO₄ and 2.5 g of KH₂PO₄ are dissolved under slow heating in a flask with 1 liter of meat water, 10.0 g of glucose, 8 ml of 1% phenol red solution and 3 ml of malachite green solution are added for setting the pH level to be 7.3±0.2 after sterilization. The medium is filtered through a membrane filter with 0.2 μm pores, then sterilized in a steam sterilizer at 121° C. for 15 minutes. Escherichia coli bacterial cells are put into the medium at an initial concentration of 10000 cells/ml of medium (into 2 sterile 500 ml vessels). The prepared solution of bacterial E. coli cells from first vessel is bottled into sterile test tubes, 5 cm³ each, the tubes are closed with wadding-gauze corks, incubated in a thermostat at 20° C. A second vessel with a solution of bacterial cells sterilized in a steam sterilizer at 121° C. for 15 minutes. The sterilized solution of bacterial E. coli cells is bottled into the sterile test tubes, 5 cm³ each, the tubes are closed with wadding-gauze corks, incubated in a thermostat at 20° C. Measurements of the intensity of light dispersed by particles in the medium are carried with an interval of 24 hours. For this reason the preliminary specimen preparation is performed: a specimen of the liquid medium with bacterial cells after fixed incubation time in a test tube is diluted 1:100 in sterile 0.9% sodium chloride solution in a 200 ml capacity, closed with a rubber cork with an aluminum rim (a 0.9% sodium chloride solution is serially produced by the pharmaceutical industry. The prepared specimen of E. coli cells is transferred by a sterile 20 ml syringe into a syringe pump and measurements of the intensity change of light dispersed by particles are performed using the PRM-6 device, described in Patent of Ukraine UA 42971C. The PRM-6 device is preliminary calibrated using spherical polysterene latexes with a refraction index n=1.56. Measurements of every specimen are performed 5-6 times with further statistical analysis of the measurement results. Between the measurements of every specimen, the hydraulic passway and optical cuvette of the device are washed with de-ionized water. For a chosen number of measurements s and a confidence probability P=0.95, Student's coefficient and limits of the confidence interval relative to a mean value are determined using formula (5).

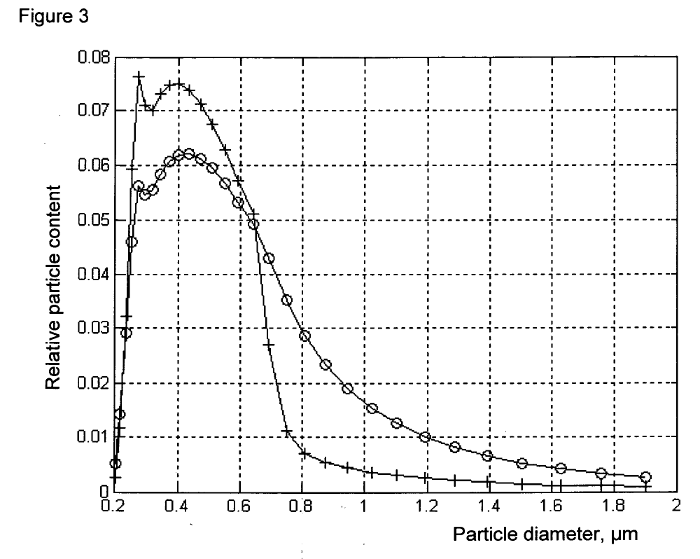

On FIG. 3, a size distribution of particles of E. coli bacterial cells' and products of disintegration of the liquid medium No. 3 (enriched medium for detection of bacteria Enterobacteriaceae) in the 0.9% sodium chloride solution are presented, wherein the size range is 0.2-2.0 μm. A curve (∘) consisting of small circles represents a suspension of particles, containing the bacterial E. coli cells from a non sterile vessel; a curve (+) consisting of plus-signs represents a suspension of particles, containing the bacterial E. coli cells from a sterilized vessel. One can observe an increase in quantity of bacterial cells particles of from a sterilized vessel in the size range of 0.25-0.65 μm and decrease in quantity of particles in the size range of 0.65-2.0 μm after 6 hours of cultivation.

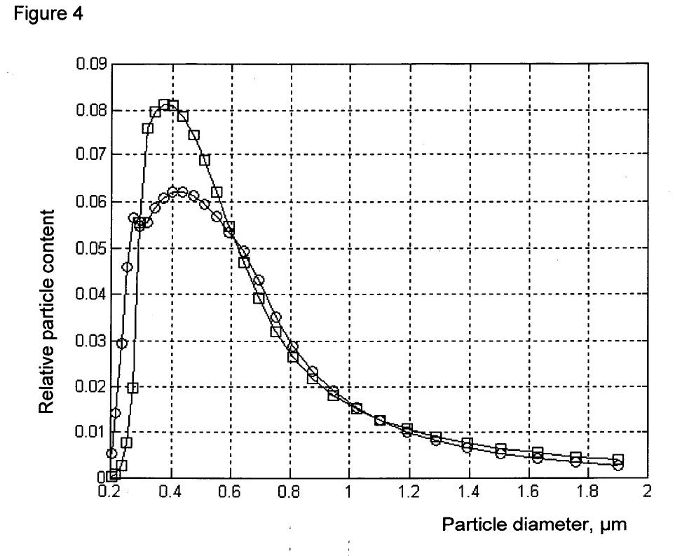

On FIG. 4, a size distribution of particles of E. coli bacterial cells' and products of disintegration of a liquid medium No. 3 (enriched medium for detection of bacteria Enterobacteriaceae) in 0.9% sodium chloride solution are presented, wherein the size range is 0.2-2.0 μm. A lower curve (∘) represents a suspension of particles, containing the bacterial E. coli cells from a non sterilized vessel; an upper curve (□) represent a suspension of particles, containing the bacterial E. coli cells from a non sterilized vessel after 24 hours of incubation. One can observe an increase in quantity of bacterial cells particles from a non sterilized vessel in the size range of 0.25-0.6 μm after 24 hours of cultivation, while quantities of particles from both specimens in the size range of 0.65-2.0 μm are equal after 24 hours of cultivation.

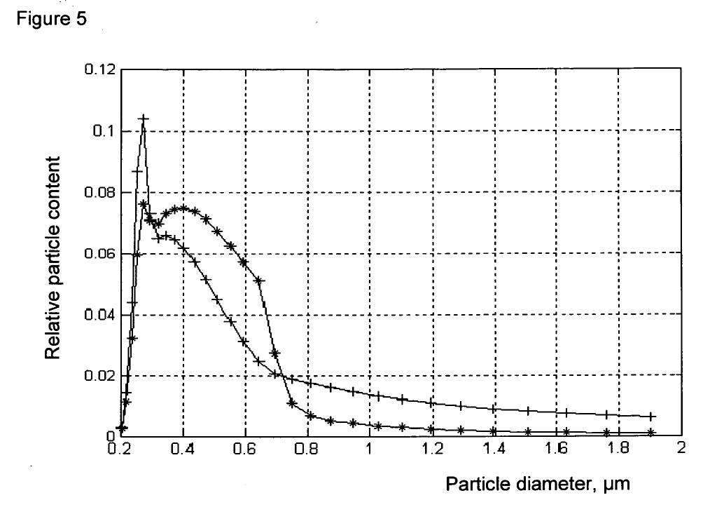

On FIG. 5, a size distribution of particles of particles of E. coli bacterial cells' and products of disintegration of the liquid medium No. 3 (enriched medium for detection of bacteria Enterobacteriaceae) in 0.9% sodium chloride solution are presented, wherein the size range is 0.2-2.0 μm. A curve (*) represents a suspension of particles, containing the bacterial E. coli cells from a sterilized vessel; a curve (+) represent a suspension of particles, containing the bacterial E. coli cells from a sterilized vessel after 24 hours of incubation. One can observe a decrease in quantity of bacterial cells particles from a sterilized vessel in the size range of 0.2-0.7 μm and increase in quantity of particles in the size range of 0.7-2.0 μm, after 24 hours of cultivation.

### Example 4

The study of Escherichia coli bacterial cells in the liquid medium No 3 (enriched medium for detection of bacteria Enterobacteriaceae) was conducted in accordance with requirements of the State Pharmacopoeia of Ukraine. For this purpose 10.0 g of dry fermentative peptone, 7.5 g of Na₂HPO₄ and 2.5 g of KH₂PO₄ are dissolved under slow heating in a flask with 1 liter of meat water, 10.0 g of glucose, 8 ml of 1% phenol red solution and 3 ml of a malachite green solution are added for setting the pH level to be 7.3±0.2 after sterilization. The medium is filtered through a membrane filter with 0.2 μm pores, then sterilized in a steam sterilizer at 121° C. for 15 minutes and equally distributed into 2 sterile 500 ml vessels. Escherichia coli bacterial cells are put into the medium of the first vessel at an initial concentration of 10,000 cells/ml of medium. The prepared solutions of liquid medium and the liquid medium containing bacterial E. coli cells are bottled into sterile test tubes, 5 cm³ each, tubes are closed with wadding-gauze corks, incubated in a thermostat at 37° C. Measurements of scattered light intensity by particles in the medium are carried with an interval of 24 hours.

For this reason the preliminary specimen preparation is performed: specimen of liquid medium with bacterial cells after fixed incubation time in a test tube is diluted 1:100 in sterile 0.9% sodium chloride solution in a 200 ml capacity, closed with rubber cork with an aluminum rim. (a 0.9% sodium chloride solution is serially produced by the pharmaceutical industry. The prepared specimen of E. coli cells is transferred by a sterile 20 ml syringe into a syringe pump and measurements of intensity change of scattered light by particles are performed using the PRM-6 device, described in the aforementioned Patent of Ukraine UA 42971C. The PRM-6 device is preliminary calibrated using spherical polysterene latexes with a refraction index n=1.56. Measurements of every specimen are performed 5-6 times with the further statistical analysis of measurement results. Between measurements of every specimen the hydraulic passway and optical cuvette of device are washed with de-ionized water. For the chosen number of measurements s and confidence probability P=0.95, Student's coefficient and limits of the confidence interval relative to a mean value are determined using formula (5).

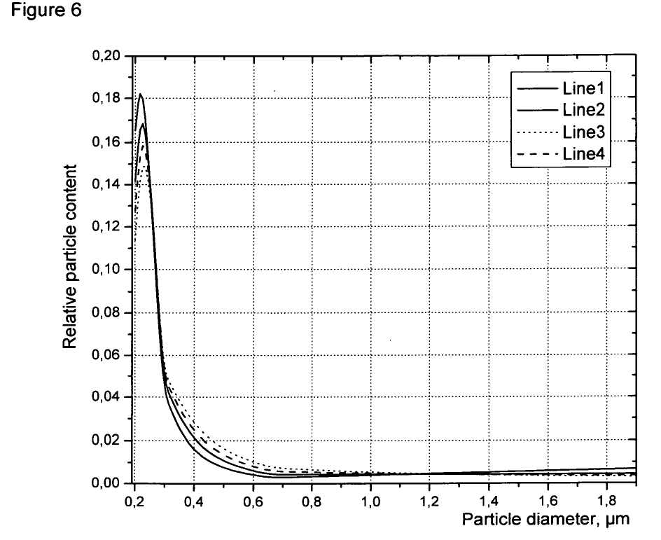

FIG. 6 depicts the relative contents of particles being products of disintegration of the liquid medium No 3 (enriched medium for detection of bacteria Enterobacteriaceae) in 0.9% sodium chloride solution, wherein the size range is 0.2-2.0 μm. Curve 1 represents suspension of particles at the beginning of cultivation process, curve 2 represents suspension of particles after 4 hours of incubation, curve 3 represents suspension of particles after 5 hours of incubation, curve 4 represents suspension of particles after 6 hours of incubation. Insignificant changes in the number of particles—products of disintegration of the liquid medium No 3-are observed during the cultivation process.

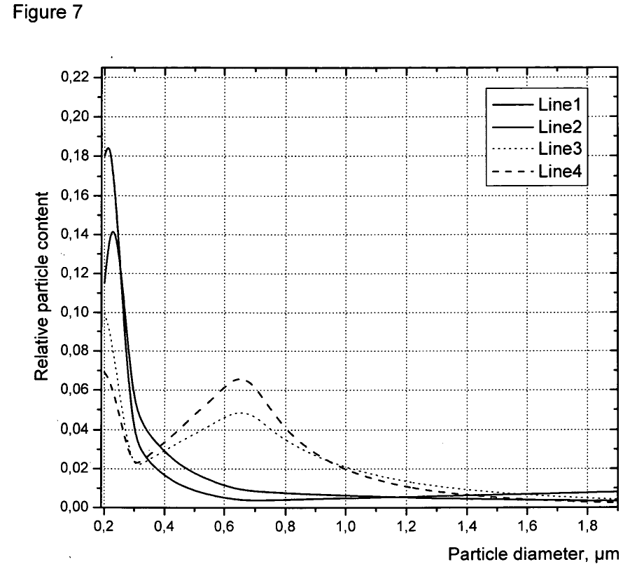

FIG. 7 illustrates the relative contents of particles—bacterial E. coli cells and products of disintegration of liquid medium No 3 (enriched medium for detection of bacteria Enterobacteriaceae) in 0.9% sodium chloride solution, wherein the size range is 0.2-2.0 μm. Curve 1 represents suspension of particles at the beginning of cultivation process, curve 2 represents suspension of particles after 4 hours of incubation, curve 3 represents suspension of particles after 5 hours of incubation, curve 4 represents suspension of particles after 6 hours of incubation. During the course of cultivation, one can observe a decrease in quantity of bacterial cells particles in the size range of 0.2-0.3 μm, and an increase in quantity of particles in the size range of 0.3-1.35 μm with a maximal number of cells with sizes of approximately 0.68 μm.

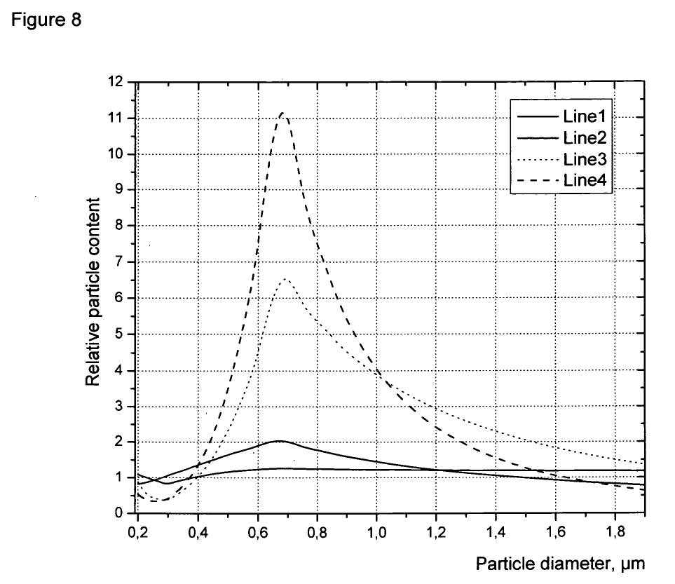

On FIG. 8, normalized distributions of relative changes of particles—bacterial E. coli cells in relation to data at the beginning of cultivation process in the liquid medium No 3 (enriched medium for detection of bacteria Enterobacteriaceae) are presented, wherein the size range is 0.2-2.0 μm. Curve 1 represents a normalized distribution of bacterial E. coli cells at the beginning of cultivation process; curve 2 represents a normalized distribution of bacterial E. coli cells after 4 hour of incubation in relation to the normalized initial distribution of these cells at the beginning of cultivation process; curve 3 represents normalized distribution of bacterial E. coli cells after 5 hour of incubation in relation to the normalized initial distribution of these cells at the beginning of cultivation process; curve 4 represents a normalized distribution of bacterial E. coli cells after 6 hour of incubation in relation to the normalized initial distribution of these cells at the beginning of cultivation process. The quantity of bacterial E. coli cells after 6 hours of incubation increased 3 times for cells with the sizes of 0.5 μm, 7 times for cells with the sizes of 0.6 μm, and 11.2 times for cells with the sizes of 0.7 μm.

### Example 5

The study of Mucor racemosus mold cells in a liquid Saburo medium in accordance with requirements of the State Pharmacopoeia of Ukraine. For this purpose 10.0 g of dry fermentative peptone and 10 g of glucose are dissolved in a flask with 1 liter of de-ionized water, for setting the pH level to be 5.6±0.2 after sterilization. The medium is filtered through a membrane filter with 0.2 μm pores, then sterilized in a steam sterilizer at 121° C. for 15 minutes. Mucor racemosus mold cells are putted into the medium of the first vessel at an initial concentration of 10000 cells/ml of the medium. The prepared solution of studied cells is bottled into sterile test tubes, 5 cm³ each, tubes are closed with wadding-gauze corks, incubated in a thermostat at 25° C. Measurements of scattered light intensity by particles in the medium are carried out after 7, 20, and 42 hours of incubation. For this reason, the preliminary specimen preparation is performed: specimen of the liquid medium with bacterial cells after a fixed incubation time in a test tube is diluted 1:100 in sterile 5% glucose solution in a 200 ml capacity, closed with a rubber cork with an aluminum rim. (5% glucose solution is serially produced by the pharmaceutical industry). The prepared specimen of mold cells is transferred by a sterile 20 ml syringe into a syringe pump and measurements of the intensity change of light dispersed by particles are performed using PRM-6 device, described in the mentioned Patent of Ukraine UA 42971C. The PRM-6 device is preliminary calibrated using spherical polysterene latexes with a refraction index n=1.56. Measurements of every specimen are performed 5-6 times with further statistical analysis of measurement results. Between the measurements of every specimen, the hydraulic passway and optical cuvette of the device are washed with de-ionized water. For the chosen number of measurements s and a confidence probability P=0.95, Student's coefficient and limits of the confidence interval relative to a mean value are determined using formula (5).

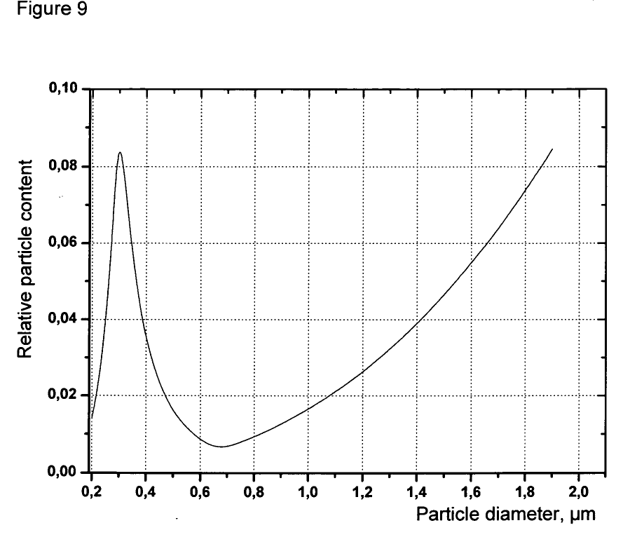

On FIG. 9, the relative content of the Mucor racemosus mold particles and products of disintegration of the liquid Saburo medium in 5% glucose solution are presented after 42 hours of cultivation, wherein the size range is 0.2-2.0 μm. After 42 hours of incubation of Mucor racemosus mold cells one can observe a size distribution curve with a clearly expressed maximum at 0.33 μm and a gradual increase in cell population in the interval of 0.7-1.9 μm.

1. A method for determining a quantity of microbiological cells in the process of their cultivation, comprising the steps of:
supplying a constant speed liquid flow containing probed objects;
probing said flow of liquid by a coherent monochromatic light and registering the intensity of light impulses dispersed by the probed objects;
calibrating by means of plotting a two-dimensional distribution of the registered impulses in a predetermined volume of spherical particles with a known index of refraction and having a radius r, as a function K(U,t,r) of the normalized amplitudes U, and durations of the impulses of the dispersed light t;
probing said flow of liquid with the probed objects by the same monochromatic coherent light and in the same volume, which was used for the calibrating;
registering impulses of the light dispersed by the probed particles at a predetermined angle in an interval from 1 to 360 degrees;
determination of a function n(r) of size distribution of the probed particles by means of registering impulses dispersed by the particles;
sorting the registered impulses for plotting a two-dimensional distribution of their amplitudes and durations in a number of sub-ranges with limits, used for the probed particles;
normalizing numeral values of the amplitudes and durations of impulses in each of said sub-ranges;
plotting a function F(U,t) based on the normalized values, which determines the size distribution of the probed objects in a size range from rₘᵢₙ to rₘₐₓ:
$$F ( U , t ) = {\int }_{{r}_{max}}^{{r}_{min}} K ( U , t , r ) n ( r ) d r ,$$
wherein the improvement is characterized by that:
said microbiological cells are cultivated in a liquid nutrient medium;
any changes in quantity of the microbiological cells and background particles are determined in the medium as follows:
diluting the nutrient medium with said microbiological cells and the nutrient medium without said microbiological cells in equal proportions in a predetermined high-purity solution;
determining a size distribution of said microbiological cells in combination with said background particles;
determining separately a size distribution of said background particles in the predetermined high-purity solution;
determining the quantity of said microbiological cells by calculation of the quantity of said particles in the nutrient medium with said microbiological cells and without them;
determining a relative content of said microbiological cells in a predetermined size interval by calculation of a ratio of a portion of said microbiological cells in the predetermined size interval to the total quantity of said microbiological cells; and
plotting time dependences of quantity changes of said microbiological cells and their relative content in the predetermined size interval during the cultivation.

2. The method according to claim 1, wherein said predetermined high-purity solution is one of the following: sodium chloride 0.9%, or 5% glucose solution, or in de-ionized water.
